SEQUENCE LISTING (1 sequence)

SEQ ID NO: 1 - DNA, 1395 bp, Cupriavidus metallidurans
  source 1..1395: mol_type = genomic DNA
gcaagtcgaacggcagcgcggacttcggtctggcggcgagtggcgaacgggtgagtaatacatcggaacgtaccctgttgtgggggataactagtcgaaagattagctaataccgcatacgacctgagggtgaaagtgggggaccgcaaggcctcacgcagcaggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatcgcgctggttaatacctggcgtggatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgggcttaacctgggaattgcgcttgtgactgcaaggctagagtgcgtcagaggggggtagaattccacgtgtagcagtgaaatgcgtagagatgtggaggaataccgatggcgaaggcagccccctgggacgtgactgacgctcatgcacgaaagcgtggggagcaaacaggattagataccctggtagtccacgccctaaacgatgtcaactagttgttggggattcattttctcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgcaagagcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcg

(19) United States
(12) Patent Application Publication
(10) Pub. No.: US 2025/0083204 A1
(43) Pub. Date: Mar. 13, 2025

(54) CUPRIAVIDUS METALLIDURANS CML2, ITS PREPARATION, AND ITS BIOREMEDIATION METHOD FOR HEAVY METAL-CONTAMINATED ENVIRONMENT

(71) Applicant: HUBEI UNIVERSITY, Wuhan (CN)

(72) Inventors: Xuejing YU, Wuhan (CN); Yong YANG, Wuhan (CN); Yuan ZHANG, Wuhan (CN); Xianhua ZHANG, Wuhan (CN); Yadong LI, Wuhan (CN); Shan WU, Wuhan (CN); Linjie LI, Wuhan (CN); Chang GAO, Wuhan (CN); Yue LU, Wuhan (CN); Tong WU, Wuhan (CN)

(73) Assignee: HUBEI UNIVERSITY, Wuhan (CN)

(21) Appl. No.: 18/588,041

(22) Filed: Feb. 27, 2024

(30) Foreign Application Priority Data
Sep. 8, 2023 (CN) 202311154136.3

(51) Int. Cl. B09C 1/10; C02F 3/34; C02F 101/20; C12N 1/20; C12R 1/01

(52) U.S. Cl. CPC B09C 1/10; C02F 3/34; C12N 1/205; C02F 2101/20; C12R 2001/01

(57) ABSTRACT

Embodiments of the present disclosure disclose a Cupriavidus metallidurans CML2, wherein the Cupriavidus metallidurans CML2 is deposited in the China Center for Type Culture Collection with a depository number CCTCC NO: M20231365, and a 16s rDNA of the Cupriavidus metallidurans CML2 has a nucleotide sequence of SEQ ID No. 1.

### CROSS-REFERENCE TO RELATED APPLICATIONS

This application claims priority to Chinese Patent Application No. 202311154136.3, filed on Sep. 8, 2023, the contents of which are hereby incorporated by reference to its entirety.

### SEQUENCE LISTING

The instant application contains a Sequence Listing which has been submitted electronically in XML format and is hereby incorporated by reference in its entirety. The XML copy, created on Jan. 5, 2024, is named “2024 Feb. 27-Sequence listing-61209-H012US00,” and is 3452 bytes in size.

### TECHNICAL FIELD

The present disclosure relates to the field of agricultural microorganisms and the prevention and control of heavy metal pollution, and in particular, an isolated Cupriavidus metallidurans CML2, a preparation including the Cupriavidus metallidurans CML2, and a bioremediation method for a heavy metal-contaminated environment using the Cupriavidus metallidurans CML2.

### BACKGROUND

With the increasing problem of environmental pollution, especially heavy metal pollution, there is a growing concern for food security. Prolonged intake of rice contaminated with excessive heavy metals (e.g., cadmium) may lead to a wide range of health problems, including osteoporosis, kidney damage, sexual dysfunction, and cancer. Limiting the accumulation of heavy metals (e.g., cadmium) in rice has therefore become an urgent issue.

Among the many ways to reduce heavy metals in rice, the bioremediation of heavy metals using endophytic bacteria in rice has become an emerging research hotspot. The endophytic bacteria are a class of microorganisms that exist in rice and have special ecological functions in the process of symbiosis with rice. For example, the endophytic bacteria can reduce the accumulation of heavy metals in rice plants through biosorption, biotransformation, extracellular precipitation, and bioaccumulation and efflux. In addition, the endophytic bacteria have shown the ability to transform heavy metals in the soil, that is, convert the heavy metals into low or non-toxicity forms through metabolic activities, thereby reducing the content of heavy metals in the soil and protecting the health of the rice growing environment.

The endophytic bacteria strains reported in previous studies, such as Stenotrophomonas maltophilia, Bacillus, and Brevundimonas diminuta, can reduce the cadmium ion concentration in the soil. However, these endophytic bacteria still exhibit only low cadmium tolerance and limited cadmium removal capability.

Therefore, it needs to provide a strain with excellent heavy metal removal ability and a bioremediation method for a heavy metal-contaminated environment.

### SUMMARY

One or more embodiments of the present disclosure provide a Cupriavidus metallidurans CML2, wherein the Cupriavidus metallidurans CML2 is deposited in the China Center for Type Culture Collection with a depository number CCTCC NO: M20231365, and a 16s rDNA of the Cupriavidus metallidurans CML2 has a nucleotide sequence of SEQ ID No. 1.

One or more embodiments of the present disclosure provide a preparation comprising the Cupriavidus metallidurans CML2.

One or more embodiments of the present disclosure provide a bioremediation method for a heavy metal-contaminated environment. In some embodiments, the method includes adding a bacterial inoculum including the Cupriavidus metallidurans CML2 to the heavy metal-contaminated environment; and incubating the bacterial inoculum in the heavy metal-contaminated environment for 1 hour to 1 week to remove heavy metal from the heavy metal-contaminated environment, wherein the Cupriavidus metallidurans CML2 is deposited in the China Center for Type Culture Collection with a depository number CCTCC NO: M20231365, and a 16s rDNA of the Cupriavidus metallidurans CML2 has a nucleotide sequence of SEQ ID No. 1.

Microbial depository information: the Cupriavidus metallidurans CML2 was deposited in the China Center for Type Culture Collection on Jul. 24, 2023, with the depository number CCTCC NO: M 20231365. The depository address is No. 299, Bayi Road, Wuchang District, Wuhan City, Hubei Province, China.

### BRIEF DESCRIPTION OF THE DRAWINGS

The present disclosure is further illustrating by way of exemplary embodiments, which is describing in detail with reference to the accompanying drawings. These embodiments are not restrictive, and in these embodiments, the same numbering denotes the same structure, wherein:

FIG. 1 is an image of the colony morphology of Cupriavidus metallidurans CML2 according to some embodiments of the present disclosure;

FIG. 2 is a phylogenetic tree of the gene sequence system of the 16S rDNA of the Cupriavidus metallidurans CML2 according to some embodiments of the present disclosure;

FIG. 3 is the growth curves of the Cupriavidus metallidurans CML2 under normal conditions and cadmium stress at 0, 400, 800, 1200, 1600, 2000, and 2400 mg/L CdCl₂ according to some embodiments of the present disclosure;

FIG. 4 is a schematic diagram illustrating the adsorption of cadmium ions by the Cupriavidus metallidurans CML2 under cadmium stress at 400, 800, 1200, 1600, 2000, and 2400 mg/L CdCl₂ according to some embodiments of the present disclosure;

FIG. 5 is a schematic diagram illustrating the effect of salt concentration, temperature, and pH on the growth of the Cupriavidus metallidurans CML2 according to some embodiments of the present disclosure;

FIG. 6 is a schematic diagram illustrating the removal effect of the Cupriavidus metallidurans CML2 on cadmium according to some embodiments of the present disclosure.

FIG. 7A-7C are schematic diagrams illustrating an effect of Cupriavidus metallidurans CML2 inoculation on rice seedling growth and cadmium remediation. FIG. 7A illustrates the effects of Cupriavidus metallidurans CML2 on rice seedling morphology; FIG. 7B illustrates the effects of Cupriavidus metallidurans CML2 on relative lengths of the root and shoot under different conditions. C: control, treated with sterile NB medium; CML2: treated with strain CML2; Cd: treated with Cd²⁺; CML2+Cd: treated with both CML2 and Cd²⁺; FIG. 7C illustrates the effects of Cupriavidus metallidurans CML2 on cadmium accumulation in rice seedling roots and shoots; data represents means±SEM; different letters (a or b) above the columns indicate significant differences between the three groups using one-way ANOVA at P<0.05; “***” indicates statistically significant differences according to a t-test, P<0.001.

### DETAILED DESCRIPTION

In order to more clearly illustrate the technical solution of the embodiments of the present disclosure, the accompanying drawings required for the description of the embodiments are briefly described below. Obviously, the accompanying drawings in the following description are only some examples or embodiments of the present disclosure, and a person of ordinary skill in the art may also apply the present disclosure to other similar scenarios without creative labor based on these drawings. Unless obviously obtained from the context or the context illustrates otherwise, the same numeral in the drawings refers to the same structure or operation.

As disclosed herein and in the claims, unless otherwise indicated by the context, the words “one,” “a”, “a kind of”, and/or “the” are not limited to the singular form and may also encompass the plural. Generally, the terms “including” and “comprising” indicate the inclusion of explicitly identified steps and elements. The listed steps and elements are not exclusive, and the method or apparatus may include other steps or elements.

One embodiment of the present disclosure provides a Cupriavidus metallidurans CML2, with a depository number CCTCC NO: M20231365, and a 16s rDNA of the Cupriavidus metallidurans CML2has a nucleotide sequence of SEQ ID No. 1

As used herein, the term “Cupriavidus metallidurans CML2” is constructed to be interchangeable with terms such as “CML2 strain”, and “rice endophyte CML2”, which may be used interchangeably to refer to the strains described in the embodiments of the present disclosure.

In some embodiments, the Cupriavidus metallidurans CML2 is an endophyte bacteria screened from rice using cadmium stress.

As used herein, the term “cadmium stress” refers to the exposure of plants to high concentrations of cadmium ions under experimental conditions to simulate cadmium-contaminated environments. The term “high concentration of cadmium ions” refers to an effective concentration of cadmium ions that causes the heavy metal content to reach a toxic level in the environment (e.g., soil or water), leading to heavy metal pollution, and poses cadmium stress on plants. In some embodiments, the concentration of cadmium ions under cadmium stress may be 400 mg/L, 800 mg/L, 1200 mg/L, 1600 mg/L, 2000 mg/L, or 2400 mg/L.

In some embodiments, the Cupriavidus metallidurans CML2 is isolated from rice.

In some embodiments, the Cupriavidus metallidurans CML2 is isolated from a stem of the rice.

The Cupriavidus metallidurans CML2 isolated and screened in the present disclosure has a high capacity for removing cadmium ions, thereby significantly reducing the concentration of cadmium ions in a heavy metal-contaminated environment and alleviating the toxic effect of cadmium ions on rice.

In some embodiments, the Cupriavidus metallidurans CML2 is capable of tolerating cadmium ions at a concentration of 2400 mg/L.

In addition, one of the embodiments of the present disclosure provides the Cupriavidus metallidurans CML2 capable of promoting rice growth and development through various means.

In some embodiments, the Cupriavidus metallidurans CML2 is capable of producing ammonium nitrogen.

Ammonium nitrogen refers to nitrogen in the form of ammonium ions present in soil, plants, and fertilizers, such as ammonium sulfate, ammonium chloride, and ammonium bicarbonate.

The ammonium nitrogen may be formed through various pathways, including ammonium nitrogen produced by ammonia oxidation and ammonium nitrogen produced by organic nitrogen decomposition.

Ammonia oxidation refers to microorganisms oxidizing ammonia (NH₃) to nitrite (NO₂⁻), and then the nitrite (NO₂⁻) is further oxidized to nitrate (NO₃⁻). In this process, the produced nitrite and the produced nitrate form the ammonium nitrogen.

Organic nitrogen decomposition refers to the decomposition of organic nitrogen in organic matter (such as plant residues, animal excreta, etc.) by microorganisms, which may release the ammonium nitrogen.

For the ammonium nitrogen produced by the Cupriavidus metallidurans CML2 in the embodiments of the present disclosure, the rice may uptake the ammonium nitrogen through its root system. The ammonium nitrogen taken up by the rice may be used as raw material for the synthesis of proteins, nucleic acids, and other important biomolecules within rice. In addition, the ammonium nitrogen in the soil is part of the soil nitrogen cycle and participates in the nitrogen metabolism process of plants and microorganisms along with other forms of nitrogen.

In some embodiments, the Cupriavidus metallidurans CML2 is also capable of producing siderophore.

The siderophore is a biomolecule, usually a protein or other organic molecule, that has a high affinity for iron ions and plays an important role in the uptake and transport of iron ions from the environment into cells. By producing the siderophore, the Cupriavidus metallidurans CML2 in embodiment of the present disclosure may promote the more effective uptake and utilization of iron ions in the soil by plants, such as rice, thereby promoting their growth and development.

One embodiment of the present disclosure provides a preparation including the Cupriavidus metallidurans CML2.

In some embodiments, the preparation may include a biopesticide, a soil remediation agent, a plant growth promoter, etc.

In some embodiments, the preparation including the Cupriavidus metallidurans CML2 may be designed as a biopesticide to protect crops from pests and diseases. The Cupriavidus metallidurans CML2 may be able to symbiotically coexist with plants in the soil, enhancing the plants' resistance to pests and diseases.

In some embodiments, the preparation including the Cupriavidus metallidurans CML2 may be designed as soil remediation agents for the remediation of metal-contaminated soil, helping to alleviate the adverse effects of heavy metals in the soil on plant growth. Introducing the Cupriavidus metallidurans CML2 is expected to improve the quality of the soil and promote vegetation restoration.

In some embodiments, the preparation including the Cupriavidus metallidurans CML2 may be designed as environmental bioremediation agents for the treatment of contaminated environments, such as industrial waste sites or metal-contaminated water bodies. By introducing such preparation, it is expected to improve the removal efficiency of metallic elements from the environment.

In some embodiments, the preparation including the Cupriavidus metallidurans CML2 may be designed as plant growth promoters, as a component of the plant growth promoter, to enhance nutrient uptake and growth rates of the plant through interaction with the plant root system.

In some embodiments, the preparation including the Cupriavidus metallidurans CML2 may be designed as a soil conditioner, which plays a role in soil improvement by improving soil structure, increasing soil organic matter content, or the like.

One of the embodiments of the present disclosure provides a bioremediation method for the heavy metal-contaminated environment. In some embodiments, the method includes adding a bacterial inoculum including the Cupriavidus metallidurans CML2 to the heavy metal-contaminated environment; and incubating the bacterial inoculum in the heavy metal-contaminated environment for 1 hour to 1 week to remove a heavy metal from the heavy metal-contaminated environment; wherein the Cupriavidus metallidurans CML2 is deposited in the China Center for Type Culture Collection with a depository number CCTCC NO: M20231365, and a 16s rDNA of the Cupriavidus metallidurans CML2 has a nucleotide sequence of SEQ ID No. 1.

As used herein, the term “bioremediation” refers to a process for treating contaminated environments or contaminated waste materials located in a specific environment. In some embodiments, the treatment involves the conversion of the waste material into less toxic material or the removal of contaminants by microorganisms or a group of microorganisms.

As used herein, the term “heavy metal” refers to metallic chemical elements with densities greater than 7 g/mL, such as mercury, cadmium, and copper.

In some embodiments, in the bioremediation method for the heavy metal-contaminated environment, the involved heavy metal includes mercury, copper, nickel, cadmium, zinc, selenium, or manganese.

In some embodiments, in the bioremediation method for the heavy metal-contaminated environment, the involved heavy metal is cadmium.

In some embodiments, the Cupriavidus metallidurans CML2 in the bioremediation method of the heavy metal-contaminated environments is capable of tolerating cadmium ions at a concentration of 2400 mg/L.

In some embodiments, the bacterial inoculum in the bioremediation method of the heavy metal-contaminated environment includes the Cupriavidus metallidurans CML2 at a concentration of 7.24×10⁶−1.5×10¹¹ CFU/mL. In some embodiments, the bacterial inoculum in the bioremediation method of the heavy metal-contaminated environment includes the Cupriavidus metallidurans CML2 at a concentration of 7.24×10⁶−1.5×10⁸ CFU/mL. In some embodiments, the bacterial inoculum in the bioremediation method of the heavy metal-contaminated environment includes the Cupriavidus metallidurans CML2 at a concentration of 1.5×10⁸−1.5×10¹¹ CFU/mL.

In some embodiments, the bacterial inoculum in the bioremediation method of the heavy metal-contaminated environment includes the Cupriavidus metallidurans CML2 at a concentration of 7.24×10⁶, 14.48×10⁶, 1.5×10⁸, or 1.5×10¹¹ CFU/mL.

In some embodiments, the bacterial inoculum in the bioremediation method of the heavy metal-contaminated environment includes the Cupriavidus metallidurans CML2 at a concentration of 1.5×10¹¹ CFU/mL.

### Embodiments

The following embodiments are some more specific illustrations of some of the embodiments associated with the above embodiments. Some of these embodiments may also be replaced or combined with corresponding elements in other embodiments to form new embodiments. The experimental processes in the following embodiments are conventional if not otherwise noted. The test materials used in the following embodiments are, if not otherwise specified, obtained by purchase from a conventional biochemical reagent company. The quantitative tests in the following embodiments were set up with three repetitions of the experiment, and the results were averaged. It should be appreciated that the following embodiments are intended to better explain the present invention and are not intended to limit the invention.

### Medium Ingredients

(1) NB medium: 3.0 g beef paste, 10.0 g peptone, and 5.0 g NaCl were weighed into a container, deionized water was added into the container to a volume of 1.0 L, and the pH of the NB medium solution was adjusted to 7.2. After high-pressure steam sterilization at 121° C. for 20min, the NB medium was obtained and stored at 4° C.

(2) NA medium: 17.0 g agar was weighed into 1.0 L NB medium. After high-pressure steam sterilization at 121° C. for 20 min, the NA medium was obtained and stored at 4° C.

(3) Ashby nitrogen-free medium: 10.0 g mannitol, 0.2 g KH₂PO₄, 0.2 g MgSO₄·7H₂O, 0.2 g NaCl, 0.1 g CaSO₄·2H₂O, 5.0 g CaCO₃, and 15.0 g agar were weighed into a container, deionized water was added to the container to a volume of 1.0 L, and the pH of the Ashby nitrogen-free medium solution was adjusted to 7.2-7.4. After high-pressure steam sterilization at 121° C. for 20 min, the Ashby nitrogen-free medium was obtained and stored at 4° C.

(4) JNFb liquid medium: 5.0 g of L-malic acid, 0.6 g of K₂HPO₄, 1.8 g of KH₂PO₄, 0.2 g of MgSO₄·7H₂O, 10.1 g of NaCl, 0.02 g of CaCl₂·2H₂O, 2.0 mL of the trace element solution (0.2 g of Na₂MoO₄·2H₂O, 0.235 g of MnSO₄·H₂O, 0.28 g of H₃BO₃, 0.008 g of CuSO₄·5H₂O, 0.024 g of ZnSO₄·7H₂O, and 1.0 L of deionized water), and 2.0 mL of bromothymol blue (0.5% aqueous solution obtained using 0.2 mol/L KOH), 4.0 mL of Fe-EDTA (1.64% aqueous solution), and 1.0 mL of vitamin solution (0.01 g of biotin, 0.02 g of vitamin B, and 1.0 L of deionized water) were added to the container, and deionized water was added into the container to a volume of 1.0 L. The pH of the JNFb liquid medium solution was adjusted to 5.8 with KOH solution. After high-pressure steam sterilization at 121° C. for 20 min, the JNFb liquid medium was obtained and stored at 4° C.

(5) JNFb solid medium: 1.5% agar was added to the JNFb liquid medium, and after high-pressure steam sterilization at 121° C. for 20 min, the JNFb solid medium was obtained and stored at 4° C.

(6) Inorganic phosphorus medium: 10.0 g of glucose, 5.0 g of Ca₃ (PO₄)₂, 0.5 g of (NH₄)₂SO₄, 0.3 g of KCl, 0.3 g of NaCl, 0.3 g of MgSO₄·7H₂O, 0.003 g of MnSO₄·4H₂O, and 0.003 g of FeSO₄·7H₂O were added into a container, deionized water was added in to the container to a volume of 1.0 L, and the pH of the inorganic phosphorus medium solution was adjusted to 7.0-7.5. After high-pressure steam sterilization at 121° C. for 20 min, the inorganic phosphorus medium was obtained and stored at 4° C.

(7) CAS test solution: 0.0605 g of chromium azurite S (CAS), 0.0729 g of cetyltrimethylammonium bromide (HDTMA), and 0.0027 g of FeCl₃·6H₂O and 50 mL of 0.1 mol/L phosphate buffer (0.296 g of NaH₂PO₄·2H₂O, 1.214 g of Na₂HPO₄·12H₂O, 0.125 g of NH₄Cl, 0.0375 g of KH₂PO₄, and 0.0625 g of NaCl) were added into a container, deionized water was added into the container to a volume of 1.0 L, and the pH of the CAS test solution was adjusted to 6.6-7.0.

(8) VF iron-deficient medium: 20.0 g of sucrose, 3.5 g of (NH₄) 2SO₄, 1.5 g of L-aspartic acid, 0.02 g of L-methionine, 0.01 g of L-histidine, 1.0 g of KH₂PO₄, 0.5 g of MgSO₄, and 0.5 g of NaCl were added into a container, deionized water was added into the container to a volume of 1.0 L. After high-pressure steam sterilization at 121° C. for 20 min, the VF iron-deficient medium was obtained and stored at 4° C.

### Embodiment 1: Isolation and Screening of the Cadmium-Tolerant Rice Endophyte CML2

Endophyte bacteria were isolated from cadmium-treated rice stems, and then the endophyte bacteria were cultivated using a high concentration of cadmium stress to screen out a strain of rice endophyte CML2 that is tolerant to 2400 mg/L CdCl₂. The specific steps are as follows:

(1) Isolation of the endophytic bacteria from rice.

The rice seedling (variety: Jinxiangyu) was selected as the experimental material, and the seedling stems (rice stems) of the Jinxiangyu rice variety were long-term exposed to a treatment of 10 μg/L CdCl₂.

Step 1: The surface of the rice stems was thoroughly washed with water to ensure its clean and free of impurities.

Step 2: Rice stems were disinfected for 1 min with 0.1% mercuric chloride solution.

Step 3: After the disinfection treatment is completed, the rice stems were rinsed with sterile water 4-5 times, and the last rinsing solution was inoculated into NB medium for incubation at 30° C. for 24 h. If there is no bacterial growth, it indicates that the sample surface was adequately sterilized; and if there is bacterial growth, steps 2-3 were repeated.

Step 4: The rice stem was sliced and these small sections of the rice stem were then carefully transferred into a sterilized mortar. 500 μL of sterile water was added to the sterilized mortar and the small pieces of the rice stem were ground. The ground liquid was taken up and inoculated into NB medium for incubation at 30° C. for 24 h.

(2) Screening of cadmium-resistant strains.

Step 1: The bacterial liquid cultured in (1) was sequentially inoculated into the NB medium with different cadmium concentrations (300, 600, 900, 1200, 1500, 1800, 2100, 2400,and 2800 mg/L) for culturing overnight at 30° C. with a shaking speed of 220 rpm.

Step 2: Bacteria that were able to grow in NB medium with 2400 mg/L CdCl₂ and grow well within 48 h were selected for bacterial strain purification using plate streaking.

Step 3: The purified strain was mixed with sterilized glycerol (a final concentration of glycerol was 30%) and stored in a refrigerator at −80° C.

A cadmium-tolerant endophytic bacterium named CML2 was finally obtained after the isolation and screening of the strain. Strain CML2 is capable of tolerating cadmium ions with a concentration of 2400 mg/L in the NB medium.

### Embodiment 2: Identification of the Rice Endophyte CML2

(1) Characterization of colony morphology.

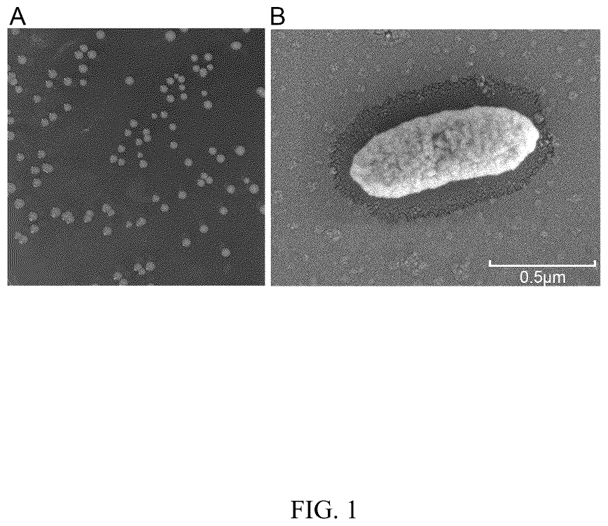

The strain CML2 was cultured on NA medium at 37° C. for 48 h and showed round colonies as shown in FIG. 1A. The diameter of the colonies was between 0.5 and 1 mm, and the surface of the colonies was smooth and moist, with a raised middle and regular edges. The color of the colonies was milky white with a yellowish tint and opaque. The bacterium exhibited a negative result in the Gram stain test.

(2) Scanning electron microscopy (SEM) observation.

The strain CML2 exhibited slightly curved rod-like structures under scanning electron microscopy, with a length of about 1-1.5 μm and a diameter of about 0.5 μm (as shown in FIG. 1B).

(3) Physiological and biochemical identification of bacteria.

API Mérieux reagent strips 20NE and ZYM were used to perform micro-biochemical and enzyme activity characterization of the strain CML2. The results were shown in Tables 1 and 2 (“+” indicating positive, “−” indicating negative). The strain CML2 was able to reduce potassium nitrate, decompose urea, and utilize dextrose, levarginine, heptaphylloside ferric citrate, 4-nitrophenyl-β-D-galactopyranoside, dextro-mannose, and potassium gluconate as carbon sources. In terms of enzyme activity characterization, the strain CML2 exhibited positive reaction to phosphatases, esterases and lipases, aryl amidases (leucine and valine), and negative reaction to proteases and glycosidases. The above identification results shown that the strain CML2 shares approximately 95% similarity to Cupriavidus metallidurans.

TABLE 1
Identification of biochemical reaction of the strain CML2

| Characterization | CML2 |
|---|---|
| Potassium nitrate | + |
| L-Tryptophan (L-Trp) | − |
| Dextrose | + |
| L-Arginine | + |
| Urea | + |
| Esculin iron citrate | + |
| Gel (cow source) | − |
| 4-Nitrophenyl-β-D-galactopyranoside | + |
| Dextrose | − |
| L-Arabinose | − |
| Dextromannose | + |
| dextro-mannitol | − |
| N-Acetylglucosamine | − |
| D-malt sugar | − |
| Potassium gluconate | + |
| Paraffinic acid | − |
| Adipic acid | − |
| Malic acid | − |
| Sodium citrate | + |
| Phenylacetic acid | − |

TABLE 2
Enzyme activity characteristics of strain CML2

| Characterization | CML2 |
|---|---|
| Alkaline phosphatase | + |
| Esterase (C4) | + |
| Esterase lipase (C8) | + |
| Lipase (C14) | + |
| Leucine arylamidase | + |
| Valine arylamidase | + |
| Cystine arylamidase | − |
| Trypsin | − |
| α-Chymotrypsin acid | − |
| Phosphatase | + |
| Naphthol-AS-BI-phosphate | + |
| Hydrolase | |
| α-Galactosidase | − |
| β-Galactosidase | − |
| β-Glucuronidase | − |
| α-Glucosidase | − |
| β-Glucosidase | − |
| N-Acetyl-β-glucosaminidase | − |
| α-Mannosidase | − |
| α-Fucosidase | − |

(4) Identification of 16S rDNA molecular of strain CML2.

The genomic DNA of endophytic bacteria in rice was extracted and used as a template for amplification of 16S rDNA via PCR.

The primer sequences were Forward Primer F27: 5′-AGAGTTTGATCATGGCTCAG-3′ and Reverse Primer R1492: 5′-ACGGTTACCTTGTTACGACTT-3′.

PCR system establishment (50 μL): 2 μL of genomic DNA, 2 μL of Forward Primer F27(10 μM), 2 μL of Reverse Primer R1492 (10 μM), 25 μL of ExTaq DNA polymerase mixed reagent, and 19 μL of sterile water.

The PCR reaction program parameters were set as follow: pre-denaturation at 94° C. for 5 min, denaturation at 94° C. for 15 s, re-annealing at 55° C. for 15 s, and extension at 72° C. for 90 s for a total of 34 cycles, and extension at 72° C. for 5 min, and the resulting PCR products was stored at 12° C.

After the PCR was finished, 5 μL of PCR product was taken and detected by 0.7% agarose gel electrophoresis, and the resulting analysis showed that the size of the target fragment was about 1.5 kb. Subsequently, the PCR products were recovered and processed, and then sent to Wuhan Jinkairui Biotechnology Co. for sequencing service. The obtained sequencing results showed that the sequence length was 1395 bp (SEQ ID No. 1:

(SEQ ID No. 1: gcaagtcgaacggcagcgcggacttcggtctggcg
gcgagtggcgaacgggtgagtaatacatcggaacgtaccctgttgtgggg
gataactagtcgaaagattagctaataccgcatacgacctgagggtgaaa
gtgggggaccgcaaggcctcacgcagcaggagcggccgatgtctgattag
ctagttggtggggtaaaggcccaccaaggcgacgatcagtagctggtctg
agaggacgatcagccacactgggactgagacacggcccagactcctacgg
gaggcagcagtggggaattttggacaatgggggcaaccctgatccagcaa
tgccgcgtgtgtgaagaaggccttcgggttgtaaagcacttttgtccgga
aagaaatcgcgctggttaatacctggcgtggatgacggtaccggaagaat
aagcaccggctaactacgtgccagcagccgcggtaatacgtagggtgcga
gcgttaatcggaattactgggcgtaaagcgtgcgcaggcggttttgtaag
acaggcgtgaaatccccgggcttaacctgggaattgcgcttgtgactgca
aggctagagtgcgtcagaggggggtagaattccacgtgtagcagtgaaat
gcgtagagatgtggaggaataccgatggcgaaggcagccccctgggacgt
gactgacgctcatgcacgaaagcgtggggagcaaacaggattagataccc
tggtagtccacgccctaaacgatgtcaactagttgttggggattcatttt
ctcagtaacgtagctaacgcgtgaagttgaccgcctggggagtacggtcg
caagattaaaactcaaaggaattgacggggacccgcacaagcggtggatg
atgtggattaattcgatgcaacgcgaaaaaccttacctacccttgacatg
ccactaacgaagcagagatgcattaggtgcccgaaagggaaagtggacac
aggtgctgcatggctgtcgtcagctcgtgtcgtgagatg).

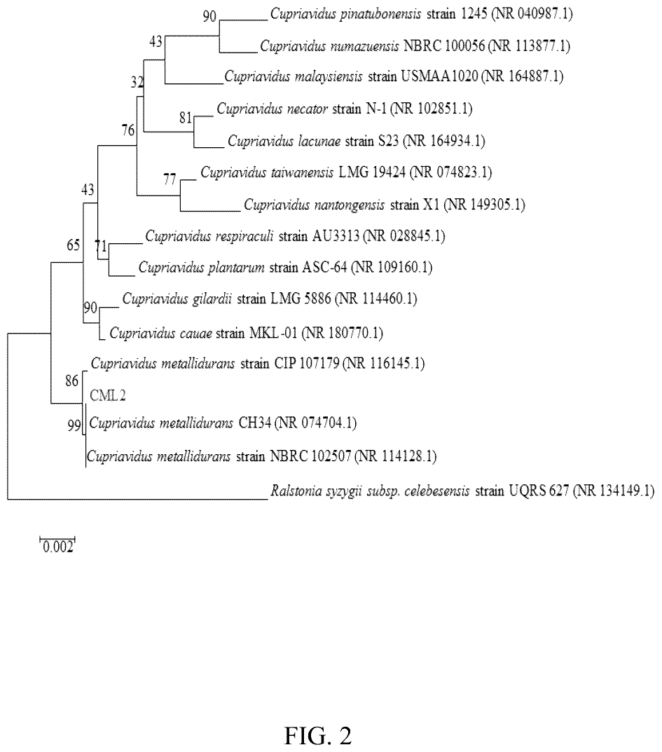

For the 16S rDNA sequence of the obtained strain, comparative analysis was performed using the Blast tool, and the results showed that the sequence exhibited 99.8% similarity to Cupriavidus metallidurans. The phylogenetic tree was constructed using the neighbor-joining method with the assistance of MEGA11 software, and the specific results are shown in FIG. 2, which indicates that CML2 clusters with Cupriavidus metallidurans CH34 and Cupriavidus metallidurans NBRC 102507. The Cupriavidus metallidurans CH34 is capable of tolerating cadmium with a maximum concentration of 3.5 mM.

Taking into account the morphological characteristics, physiological and biochemical properties of the strain, and the results of the comparative analysis of the 16s rDNA sequence, the endophyte CML2 in rice was ultimately identified as Cupriavidus metallidurans CML2 (Cupriavidus sp. CML2). The 16s rDNA sequence of this strain has been registered on the NCBI website with login number OR251471. The Cupriavidus metallidurans CML2 has been deposited in the China Center for Type Culture Collection with a depository under CCTCC NO: M 20221365 since Jul. 24, 2023. The depository address is located at No. 299, Bayi Road, Wuchang District, Wuhan City, Hubei Province, China.

### Embodiment 3: Determination of Growth Curves and Cadmium Adsorption Rates of the Cupriavidus metallidurans CML2 at Different Cadmium Concentrations

As used in this disclosure, the term “adsorption” denotes the extraction of heavy metals from contaminated sites by bacteria and the incorporation of these heavy metals into the outer cell membrane of bacteria through adsorption.

The Cupriavidus metallidurans CML2 was inoculated into 100 mL of NB medium with different concentrations (0, 400, 800, 1200, 1600, 2000, 2400 mg/L) of CdCl₂ at a ratio of 1:20, respectively. The strain CML2 was cultured at 37° C., 220 rpm, and the bacterial liquid was collected at regular intervals. The OD600 value of the bacterial liquid was measured using a PowerWave microplate reader (BioTek, USA) to reflect the growth of the bacteria. Simultaneously, 100 mL of NB medium with different concentrations (0, 400, 800, 1200, 1600, 2000, 2400 mg/L) of CdCl₂ were used as the control groups. The concentration of cadmium in the samples was determined using a flame atomic absorption spectrophotometer in accordance with the requirements of the standard GB/T 20975.6-2020 of the People's Republic of China. The cadmium content analysis was conducted utilizing the WYG2200 atomic absorption spectrometer (Waycal). Atomization of the clement was achieved through heating in a pyrolytic graphite furnace, resulting in the formation of ground state atomic vapor. This vapor selectively absorbed the characteristic radiation emitted by a hollow cathode lamp. The intensity of absorption was directly proportional to the concentration of the element present in the test solution across a specified range of concentrations. Prior to analysis, the samples underwent filtration using a 0.22 μm filter membrane. Subsequently, the changes in absorbance values at a wavelength of 228.8 nm were determined employing the air-acetylene flame method with the WYG2200 atomic absorption spectrometer (Waycal), wherein the hollow cathode lamp operated at a current of 4 mA. Based on the determination results, the growth curves of the Cupriavidus metallidurans CML2 under different cadmium concentrations were plotted, as well as the changes of cadmium concentration in different cadmium concentration medium. The cadmium removal rate was calculated using the follow formula.

$$Removal rate = ( {C}_{0} - {C}_{t} ) / {C}_{0} \times 100 \%$$

Where C₀ is the concentration of the control group (mg/L); Cₜ is the concentration of cadmium in the bacterial liquid measured by the enzyme marker (mg/L).

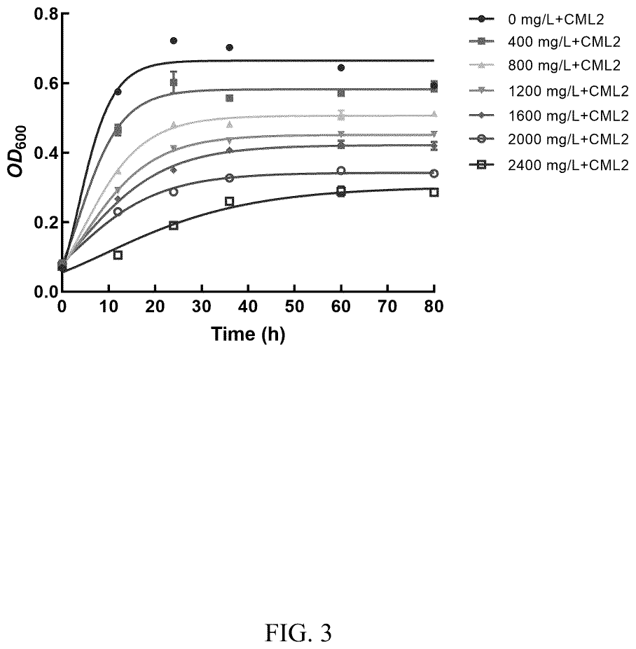

As shown in FIG. 3, in the NB medium without cadmium, the Cupriavidus metallidurans CML2 exhibited a faster growth rate and reached a stable growth period within 24 h. In the NB medium with 400 mg/L CdCl₂, however, the growth of the Cupriavidus metallidurans CML2 was inhibited, resulting in a slower growth rate. Although the Cupriavidus metallidurans CML2 was still able to reach a stable growth period within 24 h, the number of bacterial cells decreased significantly. The inhibitory effect on the growth of the Cupriavidus metallidurans CML2 was more pronounced with increasing cadmium concentration. In the NB medium with 2400 mg/L CdCl₂, the time required for the bacteria to reach a stable growth period was extended to 72 h and the number of bacterial cells declined to half of the number of bacterial cells in the NB medium without cadmium.

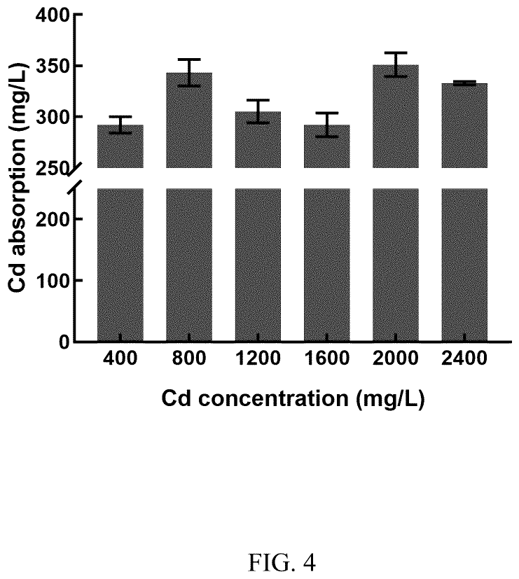

After incubation for 102 h, the Cupriavidus metallidurans CML2 reduced the cadmium concentration in the medium with 400 mg/L CdCl₂ to approximately 108 mg/L, correspondingly, a cadmium removal rate was 73%. In addition, as shown in FIG. 4, after resuspending the Cupriavidus metallidurans CML2 in the NB medium with different cadmium concentrations (400, 800, 1200, 1600, 2000, 2400 mg/L) for 5 days, a significant decrease of the cadmium concentration in the NB medium was observed. Despite the differences in cadmium removal rates under different concentration conditions, the cadmium removal was approximately 200 mg/L CdCl₂.

### Embodiment 4: Determination of the Growth-Promoting Ability of the Cupriavidus metallidurans CML2

(1) Nitrogen fixation capacity test: by PCR amplifying the nitrogen fixation enzyme genes nifD and nifH, the Cupriavidus metallidurans CML2 was inoculated into the Ashby nitrogen-free medium and the JNFb liquid medium, respectively, to observe its growth situation and determine its nitrogen fixation ability through the color change of the medium.

(2) Phosphorus solubilization ability test: the phosphorus solubilization ability of bacteria was quantitatively determined by the molybdenum antimony colorimetric method. The Cupriavidus metallidurans CML2 was inoculated into an inorganic phosphorus liquid medium, and the supernatant was obtained by centrifugation. Then, the molybdenum antimony color reagent was added to the supernatant to determine its absorbance value OD700. The phosphate content was calculated based on the standard curve drawn from the standard phosphate solution.

(3) The ammonium nitrogen production capacity test: the ability of bacteria to produce the ammonium nitrogen was quantitatively determined in a colorimetric manner with Nessler reagent. The Cupriavidus metallidurans CML2 was cultured in peptone hydroponic medium (10.0 g/L peptone, 5.0 g/L NaCl, pH 7.2˜7.4), and the supernatant was obtained after centrifugation. The supernatant was mixed with Nessler reagent for reaction and the absorbance value OD420 was measured. The amount of the produced ammonia was calculated based on the standard curve drawn with the ammonia standard solution.

(4) Plant growth hormone indoleacetic acid (IAA) production capacity test: the amount of growth hormone IAA secreted per unit volume of bacterial suspension was calculated by measuring the OD600 value of the bacterial suspension and an equal volume of Salkowski reagent (450 mL of 35% perchloric acid and 1 mL of 0.5 mol/L FeCl₃ solution)

(5) Siderophores production test: the Cupriavidus metallidurans CML2 was inoculated in the VF-Iron Deficiency medium and incubated for 3 days, then the bacterial liquid was centrifuged and the supernatant was homogeneously mixed with CAS test solution in a ratio of 1:1, and the absorbance value OD630, was measured and the production of siderophores unit SU (%) was calculated by the formula.

$$SU ( \% ) = ( Ar - As ) / Ar \times 100 \%$$

Where Ar denotes the absorbance value OD630 of a mixed solution of the supernatant of the strain and the CAS test solution, and As denotes the absorbance value OD630 of the mixture of uninoculated VF iron-deficient medium and CAS test solution.

The experimental results, as shown in Table 3, demonstrated that the Cupriavidus metallidurans CML2 exhibited certain abilities to promote plant growth and development, including the production of the ammonium nitrogen and the siderophores. However, the strain lacked nitrogenase activity and only produced a small amount of the plant growth hormone IAA. This suggests that the strain may promote plant growth and development through other mechanisms.

TABLE 3
Promoter characteristics of the Cupriavidus metallidurans CML2

| Growth-promoting properties | CML2 |
|---|---|
| Nitrogen fixation | — |
| Phosphorus solubilization (mg/L) | 1.11 ± 0.13c |
| IAA (mg/L) | 0.93 ± 0.08a |
| Ammonium nitrogen (mg/L) | 75.83 ± 9.15a |
| Ferrophilic carrier activity unit SU (%) | 22.67% |

### Embodiment 5: Determination of the minimal inhibitory concentrations (MIC) of heavy metals of the Cupriavidus metallidurans CML2

The Cupriavidus metallidurans CML2 demonstrated diverse tolerance levels to various heavy metals, as shown in Table 4. Notably, Cupriavidus metallidurans CML2 exhibited the highest tolerance to Cd²⁺ (13 mM, 2400 mg/L), Mn²⁺ (110 mM), and Hg²⁺ (0.03 mM). The tolerance hierarchy observed in Cupriavidus metallidurans CML2 was as follows: Hg²⁺<Cu²⁺<Ni²⁺<Cd²⁺<Zn²⁺<Se⁴⁺<Mn²⁺. This suggested that the Cupriavidus metallidurans CML2's heavy metal tolerance could be a basis for its potential application in the remediation of environments bearing high metal concentrations. Specifically, the pronounced tolerance of the Cupriavidus metallidurans CML2 for Cd²⁺, Mn²⁺, and Hg²⁺ indicates its potential as a candidate for mitigating these metal contaminants. The different levels of tolerance for various metals potentially signify the presence of different tolerance mechanisms, which may include absorption, accumulation, active metal efflux system, intracellular compartmentalization, production of metal-binding proteins, or detoxification by reduction or oxidation.

TABLE 4
Minimum inhibitory concentrations (MIC) values for different
heavy metals and the antibiotic resistance of strain CML2

| Heavy metal | MIC (mM) |
|---|---|
| Cadmium (Cd²⁺) | 13.1 |
| Selenium (Se⁴⁺) | 45 |
| Manganese (Mn²⁺) | 110 |
| Nickel (Ni²⁺) | 2.5 |
| Zinc (Zn²⁺) | 20 |
| Copper (Cu²⁺) | 2.0 |
| Mercury (Hg²⁺) | 0.03 |

Note that the MIC values refer to the lowest concentration of each heavy metal required to inhibit the growth of Cupriavidus metallidurans CML2.

### Embodiment 6: Optimization of Growth Conditions of the Cupriavidus metallidurans CML2

### (1) Optimum Growth Temperature

The Cupriavidus metallidurans CML2 was inoculated into 5.0 mL of NB medium and incubated at different temperatures (25° C., 30° C., 37° C., 40° C.), respectively. After incubation for 12 h, the absorbance value OD600 of the bacterium was measured and plotted.

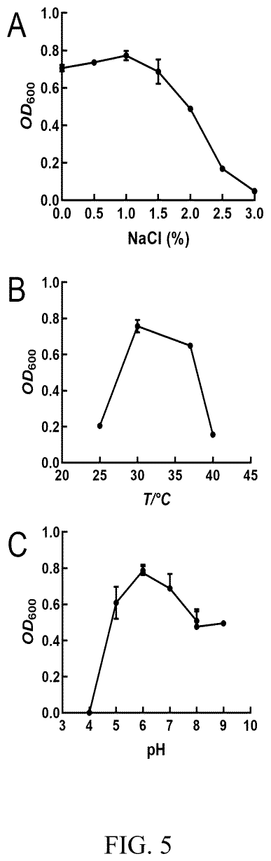

As shown in curve B of FIG. 5, the Cupriavidus metallidurans CML2 was able to grow in the temperature in a range of 25-40° C., and the Cupriavidus metallidurans CML2 has an optimum growth temperature at 30° C.

### (2) Optimal Salt Tolerance Concentration

The Cupriavidus metallidurans CML2 was inoculated into 5.0 mL of NB medium with different concentrations of NaCl (0%, 0.5%, 1.0%, 1.5%, 2.0%, 2.5%, and 3%). After incubation for 12 h at 37° C., the absorbance values OD600 of the bacteria were measured and plotted.

As shown in curve A of FIG. 5, the Cupriavidus metallidurans CML2 well at salt concentration in a range of 0%-1.5%. Bacterial growth was inhibited when the NaCl concentration exceeded 1.5%.

### (3) Optimum Growth pH

Bacterial suspensions of the Cupriavidus metallidurans CML2 were inoculated into 5.0 mL of NB medium, respectively, and cultured under different pH conditions (5.0, 6.0, 7.0, 8.0, 9.0) for 12 h. The absorbance value OD600 of the bacteria was measured and plotted.

As shown in curve C of FIG. 5, the Cupriavidus metallidurans CML2 grew well at pH in a range of 5.0-7.0. The Cupriavidus metallidurans CML2 has an optimum growth pH of 6.0.

According to the experimental results, the optimum growth temperature is 30° C., the optimum growth pH range for the Cupriavidus metallidurans CML2 is 5.0-7.0, and the optimum salt tolerance concentration range is 0%-1.5%.

### Embodiment 7: Application of the Cupriavidus metallidurans CML2 in Bioremediation of Cadmium-Contaminated Soil

Step 1: the cadmium-contaminated soil sample was sterilized. 100 mL of bacterial liquid (1.5×10¹¹ CFU/mL) was added to each 200 g of soil sample, and an equal amount of NB medium was added to each 200 g of soil sample as a control group. Each treatment was repeated in triplet

Step 2: After leaving the treated soil samples at room temperature for 5 days, the soil samples were naturally air-dried, ground, and sieved.

Step 3:5 g soil samples were separately taken, and 25 mL of DTPA leaching agent was added into the soil samples for extraction by oscillation at 25° C., 180 rpm for 2 h. The filtered extraction solution was heated at 100°° C. for 30 min, centrifuged, and filtered, and then the effective cadmium concentration of the filtered solution was measured by using graphite furnace atomic absorption spectrometer WYG2200 (Waycal) according to “National Standard of the People's Republic of China GB/T 23739-2009”.

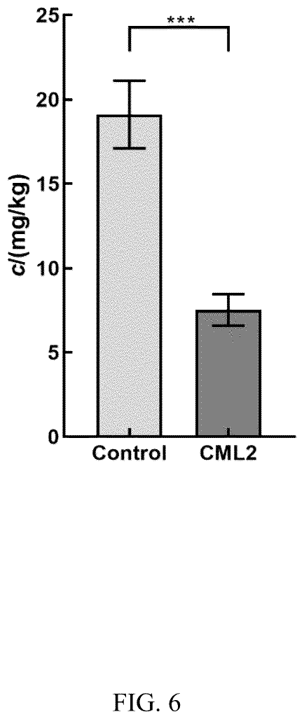

As shown in FIG. 6, compared to the control group without bacterial liquid, the Cupriavidus metallidurans CML2 is utilized to treat cadmium-contaminated soil, which may significantly reduce the cadmium concentration in the soil from an initial effective cadmium concentration of 19.11 mg/kg to about 7.52 mg/kg, with a removal rate of up to 60.62%.

### Embodiment 8: Effects of Cupriavidus metallidurans CML2 on Rice Seedling's Growth and Cadmium Remediation in Rice and Soil

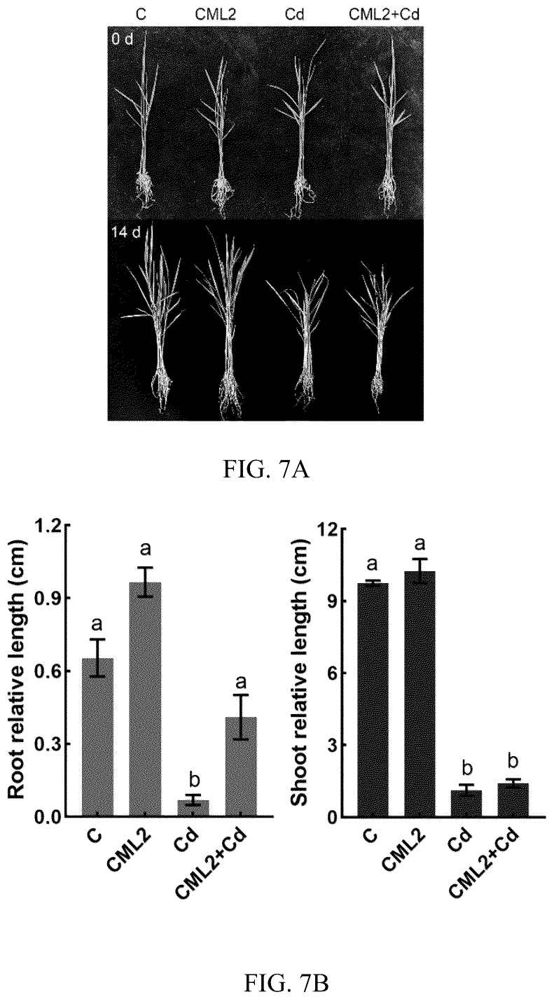

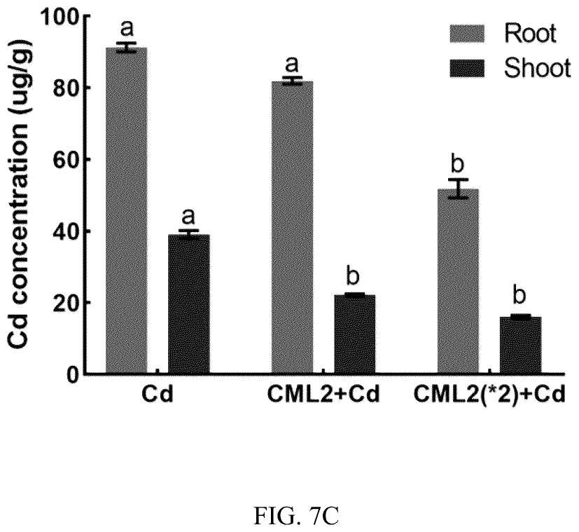

To assess the impact of strain CML2 as a facilitator for plant growth and a cadmium remediation agent, Embodiment 8 conducted an analysis on the relative lengths of rice roots and shoots as well as cadmium concentrations, under different treatments: control, Cupriavidus metallidurans CML2 (CML2), cadmium (Cd), and CML2+Cd (Simultaneous treatment with cadmium and CML2). The control group (C) consisted of rice plants cultured in a sterile nutrient solution. The results are depicted in FIG. 7A and FIG. 7B revealed after a 14-day cultivation period, the application of CML2 exhibited a promotion effect on rice root growth (48%) and shoot growth (5%) when compared to the control group. Conversely, the presence of cadmium led to significant inhibition of rice root and shoot growth, resulting in approximately 89% inhibition as compared to the control. Notably, co-treatment with Cd and CML2 demonstrated a relatively lesser degree of inhibition, with approximately 33% inhibition in root growth and 85% in shoot growth, compared to the treatment with Cd alone.

Further investigation into the cadmium levels in the roots and shoots of rice showed that the co-treatment of CML2 and cadmium resulted in lower cadmium concentrations in the roots and shoots as compared to the cadmium treatment alone. In the CML2+Cd group, with 20 mL of Cupriavidus metallidurans CML2 at a concentration of 7.24×10⁶ CFU/mL, the cadmium content in the root and shoot decreased from 92.29 μg/g and 39.75 μg/g to 81.93 μg/g and 22.24 μg/g, respectively, compared to the cadmium treatment alone. In the CML2 (*2)+Cd group, the cadmium proportions in the root and shoot decreased from 92.29 μg/g and 39.75 μg/g to 51.87 μg/g and 16.12 μg/g, respectively, compared to the cadmium treatment alone, therefore reducing the cadmium translocation rate from 43% to 31% (FIG. 7C). “(*2)” in FIG. 7C means that the treatment is 20 mL of Cupriavidus metallidurans CML2 with a concentration of 14.48×10⁶ CFU/mL. The term “cadmium translocation rate” typically refers to the movement or transfer of cadmium from roots to shoots within rice. The cadmium translocation rate can be calculated by comparing cadmium concentrations in different plant parts as follows.

$$Cadmium translocation rate = \frac{{C}_{2}}{{C}_{1}}$$

Where: C₁ is the cadmium concentration in root, C₂ is the cadmium concentration in shoot.

Embodiments of the present disclosure have at least the following technical effects: the Cupriavidus metallidurans CML2 possesses excellent cadmium resistance and removal capabilities, thus significantly reducing the cadmium ion concentration in the cadmium-contaminated environment. In addition, the Cupriavidus metallidurans CML2 is a bacterium that symbiotically associates with rice and has the ability to produce ammonium nitrogen and a Ferrophilic carrier, thereby promoting the growth and development of rice. The Cupriavidus metallidurans CML2 in the embodiments of the present disclosure is capable of tolerating higher concentrations of cadmium, while also possessing the potential to promote rice growth. Application of the bioremediation method for the Cupriavidus metallidurans CML2 described in the embodiments of the present disclosure may effectively reduce the concentration of cadmium ions in the soil, thereby mitigating the effects of heavy metal toxicity on rice and reducing the cadmium content in rice. In addition, the Cupriavidus metallidurans CML2 and the bioremediation method in the embodiments of the present disclosure may help improve the soil environment, promote sustainable agricultural development, and provide an efficient, stable, and feasible new approach to address soil heavy metal pollution and food security.

The basic concepts have been described above, and it is apparent to those skilled in the art that the detailed disclosure above is provided as an example only and does not limit the present disclosure. Although not explicitly stated here, those skilled in the art may make various modifications, improvements, and amendments to the present disclosure. Such modifications, improvements, and amendments are suggested in the present disclosure, and therefore, are within the spirit and scope of the exemplary embodiments of the present disclosure.

Moreover, the present disclosure uses specific terms to describe embodiments of the present disclosure. For example, “an embodiment”, “the embodiment”, and/or “some embodiments” refer to a feature, structure, or characteristic associated with at least one embodiment of the present disclosure. Accordingly, it should be emphasized and noted that references to “one embodiment” or “an embodiment” or “an alternative embodiment” at different locations in this disclosure do not necessarily refer to the same embodiment. In addition, certain features, structures, or characteristics of one or more embodiments of the present disclosure may be suitably combined.

In addition, unless expressly stated in the claims, the order of the processing elements and sequences, the use of numerical letters, or the use of other names as described in this disclosure are not intended to qualify the order of the processes and methods of this disclosure. While some embodiments of the invention that are currently considered useful are discussed in the foregoing disclosure by way of various examples, it should be appreciated that such details serve only illustrative purposes, and that additional claims are not limited to the disclosed embodiments, but rather, the claims are intended to cover all amendments and equivalent combinations that are consistent with the substance and scope of the embodiments of this disclosure. For example, although the implementation of various components described above may be embodied in a hardware device, it may also be implemented as a software-only solution, e.g., installed on an existing server or mobile device as described in this disclosure.

Similarly, it should be noted that in order to simplify the presentation of the disclosure of this disclosure, and thereby aid in the understanding of one or more embodiments of the invention, the foregoing descriptions of embodiments of this disclosure sometimes group multiple features together in a single embodiment, accompanying drawings, or in a description thereof. However, this method of disclosure does not imply that more features are required for the objects of the present disclosure than are mentioned in the claims. Rather, claimed subject matter may lie in less than all features of a single foregoing disclosed embodiment.

Some embodiments use numbers to describe the number of components, attributes, and it should be understood that such numbers used in the description of embodiments are modified in some examples by the modifiers “about”, “approximately”, or “substantially”. “, “approximately”, or “generally” is used in some examples. Unless otherwise noted, the terms “about,” “approximate,” or “approximately” indicates that a ±20% variation in the stated number is allowed. Correspondingly, in some embodiments, the numerical parameters used in the specification and claims are approximations, which approximations are subject to change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present specification are approximations, in specific embodiments such values are set to be as precise as possible within a feasible range.

For each patent, patent application, patent application disclosure, and other material cited in this specification, such as articles, books, specification sheets, publications, documents, etc., the entire contents of which are hereby incorporated herein by reference. Excluded are application history documents that are inconsistent with or create a conflict with the contents of this specification, as well as documents that limit the broadest scope of the claims of this specification (currently or hereafter appended to this specification). It should be noted that to the extent that the descriptions, definitions, and/or use of terms in the materials appended to this specification are inconsistent with or in conflict with the contents of what is stated in this specification, the descriptions, definitions and/or use of terms in this specification prevail. use shall prevail.

Finally, it should be understood that the embodiments described in this specification are only used to illustrate the principles of the embodiments of this specification. Other deformations may also fall within the scope of this specification. As such, alternative configurations of embodiments of the present specification may be viewed as consistent with the teachings of the present specification as an example, not as a limitation. Correspondingly, the embodiments of the present specification are not limited to the embodiments expressly presented and described herein.

1-13. (canceled)

14. A bioremediation method for a cadmium-contaminated environment comprising:
adding a bacterial inoculum comprising Cupriavidus metallidurans CML2 to the cadmium-contaminated environment; and
incubating the bacterial inoculum in the cadmium-contaminated environment for 1 hour to 1 week to reduce cadmium from the cadmium-contaminated environment; wherein
the Cupriavidus metallidurans CML2 is deposited in the China Center for Type Culture Collection with a depository number CCTCC NO: M20231365 on Jul. 24, 2023, and a 16s rDNA of the Cupriavidus metallidurans CML2 has a nucleotide sequence of SEQ ID No. 1.

15. The bioremediation method according to claim 14, wherein the reducing cadmium from the cadmium-contaminated environment is to reduce cadmium content in cadmium-containing soil or wastewater.

16. The bioremediation method according to claim 14, wherein the reducing cadmium from the cadmium-contaminated environment is to reduce a stress on rice growth caused by cadmium ions or to reduce the cadmium content in rice.
